(19) United States
(12) Patent Application Publication
(10) Pub. No.: US 2012/0220878 A1
(43) Pub. Date: Aug. 30, 2012

(54) Blanching Device for Use in Evaluating Skin Condition

(76) Inventors: Alan Sullivan, Leixlip (IE); Michael Sullivan, Blackrock (IE)

(21) Appl. No.: 12/996,561

(22) PCT Filed: Jun. 8, 2009
(86) PCT No.: PCT/EP2009/004105

§ 371 Date: May 11, 2012

(30) Foreign Application Priority Data
Jun. 6, 2008 (IE) S2008/0464

(51) Int. Cl. A61B 5/103; A61B 6/00

(52) U.S. Cl. 600/476; 600/587

(57) ABSTRACT

A blanching device is described for use in evaluating skin condition. The blanching device has a window which is pressed against the skin, and retracts when a pre-determined pressure is reached to allow blood to re-enter the blanched region of skin. There is further described systems to evaluate skin condition using a blanching device, based on light scattered from the blanched region.

### FIELD OF THE INVENTION

This invention relates to a blanching device for use in evaluating skin condition, and to a system incorporating such a device.

### BACKGROUND OF THE INVENTION

The first sign of a pressure ulcer is as an area of persistent redness which may be visible on some skins and not others depending upon their pigmentation. This persistent redness is due to soft tissue being compressed for a long period of time between a bone and a firm surface such as a mattress causing an interruption of the blood supply which the body corrects by means of a temporary elevation of the blood flow to the area.

Clinically, nurses are able to detect early pressure ulcers by the redness of the skin by compression of a reddened area with a finger to ‘blanch’ the area and assessing the rate at which the blanch disappears. If the reddened area of skin blanches and then returns to red on release of pressure within a predetermined time, this is called blanchable erythema, and is not generally considered to constitute a serious change in skin health by nurses.

In cases where the redness or erythema persists after compression with a finger; called non-blanchable erythema, this is an indication that damage has occurred, due to a more severe interruption of the blood supply and inflammation, either due to excessive pressure or lower pressures acting over a longer duration, bringing about a more serious change in the blood circulation.

The ‘finger blanch’ test is problematic in that it requires much subjective judgment on the part of the nurse or clinician, and the variances of skin pigmentation and condition make it difficult to observe changes over time. An existing laboratory based technique has been described to monitor and characterise skin redness using tissue reflectance spectroscopy (TRS). A fibre optic probe is used to deliver light and detect back-scattered light from the superficial dermis. The back-scattered light can be analysed for spectral components that have interacted with the blood. Using appropriate analysis algorithms it is possible to determine indices of blood content and oxygenation for the superficial skin vasculature.

Although these procedures allow precise and reliable measurements, their current use is limited to static measurement of quantities related to blood content in the skin. This does not necessarily relate to the seriousness of damage, which is established using manual methods by expelling blood from an area and observing the speed of re-colouration using the human eye. This dynamic measurement of the skin's response to blanching, is a more accurate indication of the degree of damage caused to the microcirculatory system, because the damaged area contains a higher degree of extravascular blood than an undamaged area.

Response to blanching has been identified as having value both in identifying erythema, and in differentiating between different types of erythema: non-blanching erythema exhibits a fast recovery of blood content after blanching, whereas reactive hyperemia (blanching erythema) exhibits a slow response.

Prior art exists, employing light emitting diodes to illuminate the skin, and measuring the backscattered signal using photodiodes, to deduce blood content. This technology is well described in photoplethysmography, and pulse oxymetry.

A previous device (WO0060349) used this approach to provide a rapid and non-invasive diagnostic apparatus and method for assessing and differentiating patency of and damage to tissue microcirculation, for instance non-blanching and blanching erythema of the skin, by the measurement of blood content and the response of tissue to blanching. This device created a blanched area of skin by means of sliding a raised edge over the skin to produce a white streak, which could then be monitored as it recovered to the normal level of skin blood content.

This technique suffered from the following problems:

1) The sliding motion required for the blanching edge meant that the region of skin under the sensor after the slide was different from the region of skin under the sensor before the slide. Variations in skin blood content could not therefore be attributed reliably to dynamic variations post-blanching, but may in part be attributable to regional variations in the skin. For example, sliding from a red area of skin to a less red area of skin may create ambiguous readings, possibly interpreted as a failure to recover totally from the blanching action.

2) The sliding device proved to suffer from poor inter-user reliability, as different users would slide in a different way. Holding the probe at an angle deviating even slightly from perpendicular to the skin, for example, would give an incomplete blanch.

3) Concerns were raised over the possible abrasive damage done to friable skin (common in the groups at risk of pressure ulcers). In clinical trials, some cases of suspected damage were observed.

Another device has been developed (E21594399) comprising an automated blanch induced by a pneumatic device. This purportedly overcame some of the reliability problems of the former device, by automating the blanch process. However, the commercial viability of the device is compromised by the expense of controlling an automated pneumatic system, and complicated in terms of provision for infection control.

### SUMMARY OF THE INVENTION

According to one aspect the invention provides a blanching device for use in evaluating skin condition, comprising at least one window carried at the lower end of a support for pressing the window downwardly against the skin to blanch a region of the skin, the window being yieldably connected to the support such that upon reaching a pre-determined blanching pressure the window rapidly retracts upwardly relative to the support to relieve the pressure on the skin and allow blood to re-enter the blanched region.

Preferably, the window is provided having a convex shape projecting downwardly from said support.

In one embodiment the window forms part of a retractable component and the support and the component have interfering formations which resist retraction of the component until the predetermined blanching pressure is reached at which point the resistance is overcome so that the formations snap past each other to allow the component to retract.

Preferably, said interfering formations comprise an angular détente provided on the support, and an annular collar provided on the retractable component, the annular collar arranged to abut said angular détente.

Preferably, said angular détente is provided on an interior wall of said support, said retractable component arranged to retract into the interior of the support.

In another embodiment the edges of the window are flexibly connected to the support such that the window retracts by rapidly inverting to a concave shape when the predetermined blanching pressure is reached.

Preferably, said support comprises an opaque shroud operable to shield the region of the skin from ambient light.

Preferably, a resilient seal portion is provided at the lower end of the support.

The use of a resilient seal portion, formed from e.g. natural or artificial rubber, allows for a good light-proof seal to be formed about the boundary of the region of skin under test.

Preferably, the blanching device comprises a plurality of adjacent windows provided at the lower end of the support for blanching a plurality of adjacent regions of skin.

A further problem with the prior devices is that the optical signals are highly sensitive to the angle with which the devices are placed on the skin, leading to a possible source of error, and user training issues.

According to a further aspect the invention provides a system for use in evaluating skin condition, comprising means for momentarily pressing on the skin to blanch a region of the skin and then allow blood to re-enter the blanched region, the apparatus further including at least one light emitter to direct light onto the blanched region of skin and at least one light detector to monitor said light after scattering from the blanched region of skin, wherein the light emitter and light detector direct light onto, and receive light from, the blanched region of skin substantially along a common axis which is non-normal to the blanched region of skin so as to substantially eliminate specular reflections from the detected light.

In a preferred embodiment the light is directed onto the blanched region of skin through a window and the common axis is non-normal to the blanched region of skin after refraction by the window, the common axis also being non-normal to the interior surface of the window.

Preferably, the light emitter emits light having a wavelength substantially within the range 450 nm-900 nm, most preferably within the range 800 nm-880 nm.

Preferably, the light emitter is operated at a switching frequency. This allows for the reduction of noise due to ambient light.

Preferably, the switching frequency is substantially within the range 10 Hz-100 kHz.

Preferably, the system comprises two or more light emitters having different wavelengths of emitted light, said wavelengths substantially within the range 450 nm-900 nm.

Preferably, the system comprises first and second light emitters, wherein the wavelength of the light emitted from the first and second light emitters respectively are substantially within one of the following pairs of ranges: 420 nm-450 nm and 520 nm-580 nm; 600 nm-680 nm and 800 nm-880 nm; or 520 nm-580 nm and 600 nm-680 nm.

According to a still further aspect the invention provides a system for use in evaluating skin condition, comprising means for momentarily pressing on the skin to blanch a region of the skin and then allow blood to re-enter the blanched region, at least one light emitter to direct light onto the blanched region of skin, and at least one light detector to monitor said light after scattering from the blanched region of skin and provide a generally exponential rising signal corresponding to the re-entry of blood into the blanched region, the apparatus further including processing means for analysing the signal into a plurality of components having different time constants, and calculating the proportions of the components in the total signal.

Preferably, the processing means is operable to analyse the generally exponential rising signal Z corresponding to the re-entry of blood into the blanched region as the sum of three exponential curves A, B, and C such that:

Z=p.A+q.B+r.C

where p, q and r are coefficients, and p+q+r=1.

Preferably, the processing means is operable to select A such that the time constant for A is substantially within the range 0.1-1 second, most preferably about 0.4 seconds.

Preferably, the processing means is operable to select B such that the time constant for B is substantially within the range 3-12 seconds, most preferably about 6 seconds.

Preferably, the processing means is operable to select C such that the time constant for C is substantially within the range 0.75-2 seconds, most preferably about 1.5 seconds.

Preferably, the processing means is operable to calculate a Hyperaemia Indication (HI) value, such that:

(HI)=q/(p+q)

Preferably, the processing means is operable to calculate an Ischaemic Damage Indication (IDI) value, such that:

(IDI)=p/(p+q)

It will be understood that the term “light” as used herein can include the range of electromagnetic radiation wavelength comprising both the visible and near visible spectrum.

It will also be understood that expressions such as “lower” and “downwardly” are used herein for convenience and ease of explanation, and refer to the normal orientation of the device in use, as shown in FIGS. 1 and 2 for two embodiments of devices according to the invention. However, the device can be used in any convenient orientation.

Using the invention one can make a rapid non-invasive assessment of the tissue status, particularly with respect to the quality of microcirculation. This information allows an assessment to be made of skin condition, identifying erythema associated with early pressure ulcers, susceptibility to damage caused by mechanically-induced ischaemia. The invention also allows differentiation between healthy dermal or sub-dermal tissue and bruised or inflamed tissue.

The invention is particularly effective in, but not limited to, risk assessment and/or early detection of pressure ulcers. Any medical condition or disease which can be assessed by measuring the competence of microcirculatory supply and drainage will also be a potential application for the present invention, for example, diabetes, vascular insufficiency, leg ulcers as well as monitoring the status of tissue flaps and skin grafts.

### BRIEF DESCRIPTION OF THE DRAWINGS

Embodiments of the present invention will now be described, by way of example, with reference to the accompanying drawings, in which:

FIG. 1 is an enlarged schematic cross-sectional view of a blanching device according to a first embodiment of the invention.

FIG. 2 is an enlarged schematic cross-sectional view of a blanching device according to a second embodiment of the invention.

FIG. 3 is a graph of the preferred pressure/time profile for the blanching devices of FIG. 1 and FIG. 2.

FIG. 4 is a block diagram of a system incorporating a blanching device as shown in FIG. 1 or FIG. 2.

FIG. 5 is a flow diagram of the operation of the apparatus of FIG. 4.

FIG. 6 is a representation of the recovery curve of skin blood content versus time, post blanch, showing the total signal modeled as the sum of 3 exponential curves.

FIGS. 7(a) and 7(b) are schematic front and side views respectively of an alternative blanching cap for the device of FIG. 1.

### DESCRIPTION OF PREFERRED EMBODIMENTS

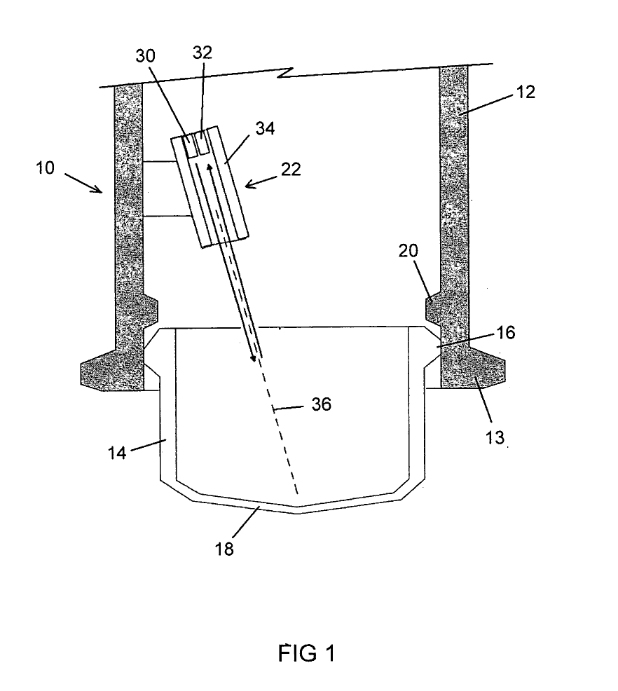

Referring to FIG. 1, a blanching device 10 comprises a transparent cap 14 supported at the lower end of a hollow opaque shroud 12. The cap 14 has an annular collar 16 which engages the interior walls of the shroud 16 with just sufficient friction to retain the cap in place. The lowest part of the cap 14 forms a window 18 whose outer surface has a generally convex, i.e. outwardly and downwardly bulging, shape.

The shroud 12, of which only the bottom part is shown, serves as a handle which allows the window 18 to be pressed downwardly against a patient's skin (not shown) either manually or using an instrument. When such pressure is first applied, retraction of the cap 14 upwardly into the shroud 12 is resisted by the collar 16 which meets an annular detente 20 on the interior wall of the shroud. This allows the window 18 to be pressed firmly against the skin to blanch the skin under the window.

However, when the pressure of the window 18 on the skin reaches a pre-determined blanching pressure, the annular collar 16 “snaps” upwardly past the detente 20, by resilient deformation of the shroud and/or the cap. This allows the cap 14, and correspondingly the window 18, to retract rapidly up into the shroud 12 to relieve the pressure on the skin and allow blood to re-enter the blanched region. The cap 14 is fully retracted when the lower end 13 of the shroud 12 comes into contact with the skin, which defines an end point for the retraction and also stabilises the blanching device post-blanch. The rate of re-flow of blood into the blanched region is monitored by a sensor unit 22 to be described below, the shape of the blood recovery signal indicating the state of microcirculation of the underlying tissue.

The blanching device 10 achieves two objects: firstly, the skin is momentarily blanched with a pre-defined pressure. Secondly, immediately after the retraction of the cap 14 the blanched region of skin is shielded from ambient light by the opaque shroud 12. The lower end 13 of the shroud 12, i.e. the part of the shroud which contacts the skin, may be made of natural or artificial rubber to accommodate undulations in the skin surface and provide a good light-proof seal with the skin. For reasons of hygiene and disease control, the entire cap 14 is removed from the shroud 12 and disposed after each use, and is replaced each time with a fresh cap 14. Alternatively, it would be possible to design the cap so that just the lower end of the cap 14 including the window 18 is replaceable, so retaining the main body of the cap for re-use.

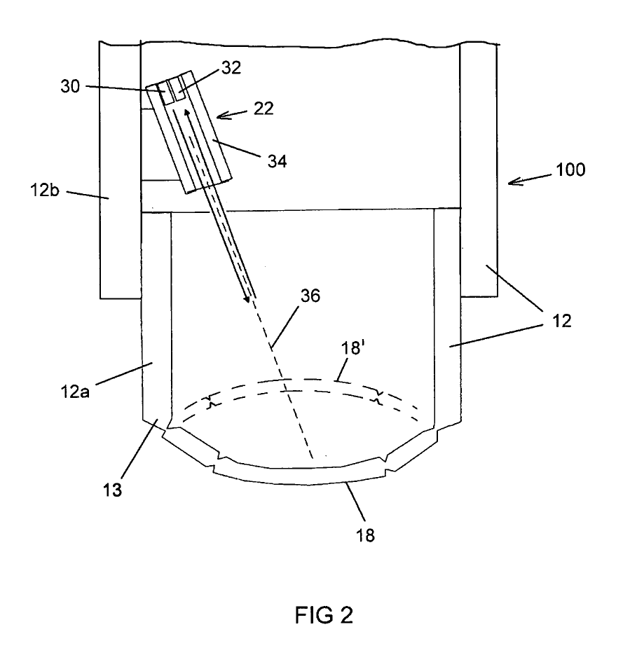

FIG. 2 shows an alternative form of blanching device 100. Here a generally convex transparent window 18 is integral with the lower end 13 of the shroud 12 and comprises a ‘pop’ mechanism, designed to yield at a given pressure, in a similar manner to pop-up lids on jam jars. The edges of the window 18 are flexibly connected to the lower end 13 of the shroud such that the window retracts into the shroud by rapidly inverting to a concave shape, as indicated by the dashed lines 18′, when a pre-determined blanching pressure is reached. This rapidly removes the pressure on the skin and allows blood to re-enter the blanched region.

As before, the shroud 12 is opaque, apart from the window 18. In this case the entire bottom part 12a of the shroud is removed from the main body 12b of the shroud after use and replaced with a fresh part. Again, the rate of re-flow of blood into the blanched region is monitored by a sensor unit 22.

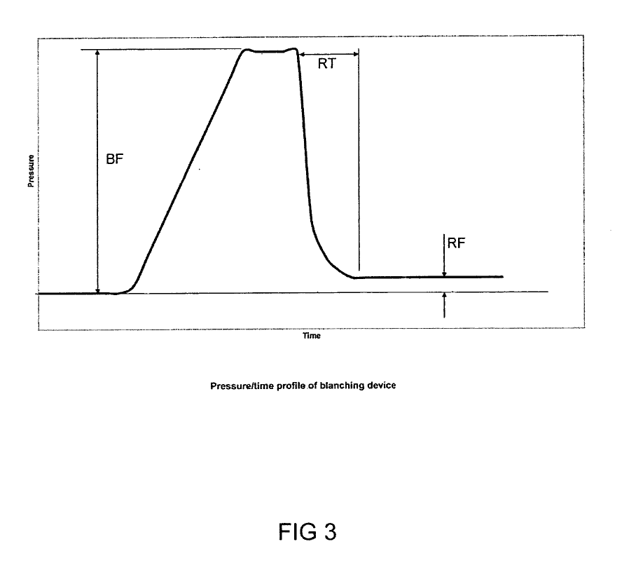

Preferably, in use of the blanching device, the pressure applied to the skin by the window 18 follows the profile shown in FIG. 3.

Initially, zero pressure is applied, during which time blood parameters appear as baseline. Pressure is then ramped up to a reproducible and consistent blanching level (BF), which is preferably in the range 50 mmHg to 350 mmHg, most preferably 120 mmHg to 150 mmHg, depending on the geometry of the blanching device, and other variables.

After blanch is achieved, the pressure on the skin “snaps” off, reducing to a lower residual level of pressure (RF). The residual level RF should be consistent and reproducible, and is preferably in the range 0 mmHg to 20 mmHg, most preferably 1 mmHg to 5 mmHg, depending on the geometry of the blanching device. The time taken to reduce to the residual level RT should also be consistent and reproducible, and is preferably in the range 0 seconds to 1 second (most preferably 0-0.25 seconds).

In the prior art, a common problem was sensitivity to the angle of placement of the device on the skin. Reflected light intensity would vary significantly with the angle of incidence. The predominant artifact causing this problem is the collection of specular reflections. Diffuse reflections emanate at a much more uniform intensity at different angles of reflection, whereas the angle of specular reflections is equal to the angle of incident light. This problem also rendered the prior devices susceptible to errors due to variations in shininess of skin due to wetness, greasiness or other factors, being interpreted as variations in blood content. The construction and placement of the sensor unit 22 is designed to mitigate this problem.

The sensor unit 22 comprises at least one light emitter 30 to direct light onto a region of skin blanched by the window 18, and at least one light detector 32 to monitor the intensity of light from the emitter 30, after scattering from the blanched region of skin, during the re-flow of blood into the blanched region after the window pressure is relieved. The light emitter(s) 30, which may be LEDs, and the light detector(s) 32, which may be photodiodes, are surrounded by a baffle tube 34 to limit the angle of sensitivity of the sensor unit 22 and make it directionally sensitive. Thus, due to the baffle tube 34, the light emitter(s) and detector(s) 30, 32 direct light onto, and receive light from, the region of blanched skin substantially along a common axis 36.

The sensor unit 22 is offset from the centre line of the cap 14 and has its axis 36 angled towards the centre of the window 18 so that the axis 36 is non-normal to both the interior surface of the window 18 and, after refraction by the window, the surface of the region of blanched skin.

Preferably the axis 36 makes an angle of from 50-70 degrees with the interior surface of the window 18 and with the surface of the blanched skin. This means that unscattered specular reflection from either surface reflects at such an angle as to miss the sensor unit 22, thereby substantially eliminating the detection of specular reflections from either surface and so rendering the device relatively insensitive to the angle of application to the skin. (It will be understood that the directionally sensitive sensor unit 22 constructed as above can be used to substantially eliminate specular reflections in blanching devices which do not include a window, i.e. where the light falls directly on bare skin, as in the device of WO0060349. In that case the unit 22 would be orientated such that its axis 36 fell at an acute angle onto the blanched skin revealed by the sliding edge.)

Where a single light emitter 30 is used, it preferably has a wavelength in the range 450 nm-900 nm, most preferably 800-880 nm. Reduction of noise due to ambient light may be overcome by switching the light emitter repeatedly on and off at high frequency, e.g. between 10 Hz and 100,000 Hz, and recording the differences in signal values between when the light emitter is on and off.

Reduction of artifacts due to chromophores other than haemoglobin may be achieved by employing two or more light emitters with different wavelengths of light, with differing extinction coefficients for haemoglobin. These wavelengths may be anywhere in the range 450 nm to 900 nm, but are preferably within the following pairs of ranges: 420-450 nm and 520-580 nm 600-680 nm and 800-880 nm 520-580 nm and 600-680 nm

Immediately following blanching of the skin, using the sensor unit 22 measurements are made from the illuminated skin using spectrophotometric, photoplethysmographic or pulse oxymetric techniques well known in the art.

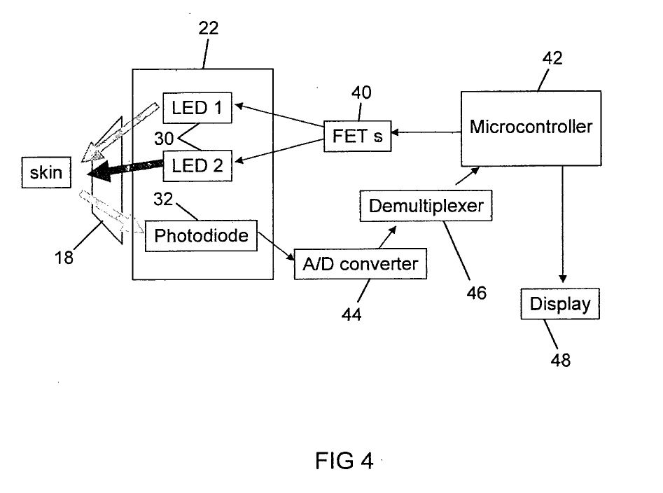

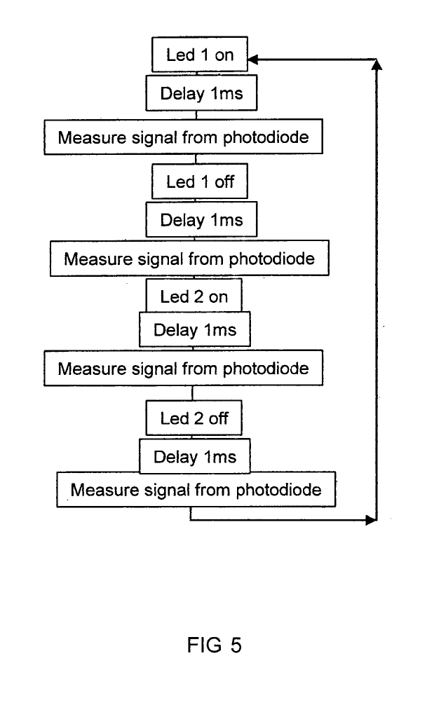

FIG. 4 is a block diagram of a system incorporating a blanching device as shown in FIG. 1 or FIG. 2. In this case the system is assumed to include two light emitters 30 (LED 1 and LED 2) emitting different wavelengths and a single photodiode light detector 32. During operation of the system, the LEDs are switched on and off by field effect transistors (FETs) 40 under the control of a programmed microcontroller 42, e.g. according to the scan loop shown in FIG. 5. This scan loop commences when the blanching device is switched on, or at least when pressure is first applied to skin by the window 18, and continues through retraction of the window and for a predetermined period afterwards, e.g. 20 seconds. The signal from the photodiode 32, which contains data relating to both LEDs, is A/D converted at 44 and the data relating to each LED, i.e. the changing amplitude with time of the light scattered from the blanched region of skin for each wavelength, is separated out in the demultiplexer 46. The microcontroller 42 calculates various parameters of the tissue based on the demultiplexed signals to allow evaluation of the microcirculatory damage adjacent to the tissue surface. These parameters are displayed on a display device 48.

For example, the microcontroller may measure the level and ratio of oxygenated to deoxygenated blood in the blanched region, which allows identification of bruised or necrotic skin areas as distinct from erythema by analysing the time variation of light attenuation data during the blanching process. Oxyhaemoglobin and deoxyhaemoglobin have certain differences in absorption spectrum, notably in the red to near infra-red region. An isobestic point at 810 nanometers exists, where oxyhaemoblobin and deoxyhaemo-globin have the same value of absorption. A point in the red range, for example 650 nanometres, can be chosen such that oxyhaemoglobin has a substantially lower value for absorption than deoxyhaemoglobin. Using known techniques, for example in the form of pulse oximeters, it is possible to calculate blood oxygenation based on a ratio, difference or other comparison of absorption values at 600-650 nm with the absorption value at an isobestic point, e.g. 810 nm.

Although the above system can perform measurements based on techniques known in the art, we have found that tissue condition can be evaluated using a new technique.

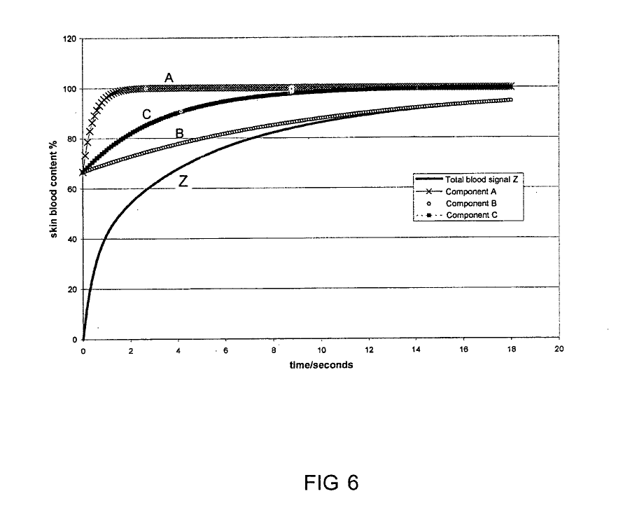

Referring now FIG. 6, the exponential curve labeled Z shows the recovery of skin blood content to a nominal baseline level following a blanch. The curve Z is the output of the photodiode 32 for one particular wavelength of light after filtering and smoothing. Variations in this curve exist in apparently haphazard ways between individuals and between different parts of the body. Past attempts to relate features of this curve to skin pathology have had limited success.

It has now been found that greater success is achieved if the total curve Z is analysed as the sum of 3 exponential curves, here represented as components A, B, and C, each with different fixed time constants. Thus:

Z=p.A+q.B+r.C

where p, q and r are coefficients defining the proportions of the three components A, B and C respectively in the total signal Z, and p+q+r=1.

Values for the time constants for A, B, and C that have been shown to be useful in distinguishing different skin pathologies are:

A: 0.1-1 second, preferably 0.4 seconds.

B: 3-12 seconds, preferably 6 seconds.

C: 0.75-2 seconds, preferably 1.5 seconds.

Experimentation with different forms of artificially-induced erythema has shown that different skin conditions exhibit these 3 components in different proportions.

In normal skin at rest, some capillaries are closed by the action of pre-capillary sphincters, some are bypassed by the opening of arteriolar-venular shunts, and some are engorged. Capillaries are arranged in different layers or plexi to supply different levels of tissue.

Different plexi of capillaries in different conditions as described in the preceding paragraph exhibit different behaviour in terms of reflow dynamics.

The magnitude of the B component, the slowest recovery, is greatest in the extremities, and varies much between body sites and between healthy individuals. This variation confounded previous attempts to analyze the recovery curve in terms of the time constant of a single exponential, and dominated calculations of time constant.

The C component (medium time constant) is also present in normal skin, and represents the condition of normally-perfused capillaries in the papillary loops of the dermis.

The A component (shortest time constant) indicates an inflammatory response caused by damage to the dermis.

Reactive hyperaemia, a harmless elevation of blood supply to redress short-term metabolic deficits, is frequently seen as a red patch on the skin. It may be induced, for example, by a short period of sitting. This harmless type of erythema can be distinguished from genuine ischaemic damage, since it is accompanied by an elevation of the B component in the skin to produce redness, while the A component remains relatively low. In contrast, an early pressure ulcer exhibits a pronounced elevation in the A component.

Both of these changes would previously have been observed as a shortening of the time constant of the recovery curve. By modeling as components A, B, and C the following parameters can be thus extracted from the recovery curve:

Overall Blanch Recovery (OBR)=Z=p.A+q.B+r.C

Hyperaemia Indication (HI)=q/(p+q)

Ischaemic Damage Indication (IDI)=p/(p+q)

Very low HI suggests poor vascularity, and predisposition to pressure ulcers. High IDI suggests early pressure ulcer formation. High baseline blood content but low OBR indicates haemmorrhage. Haemmorrhage+IDI indicates non-blanching erythema.

Accordingly, in a preferred embodiment the microcontroller 42 analyses the total blood signal from the photodiode for one of the wavelengths of light, from LED 1 or LED 2, to derive the components A, B and C, and calculates and displays on the display 48 the proportion coefficients p, q and r as well as, if desired, the values for OBR, HI and IDI.

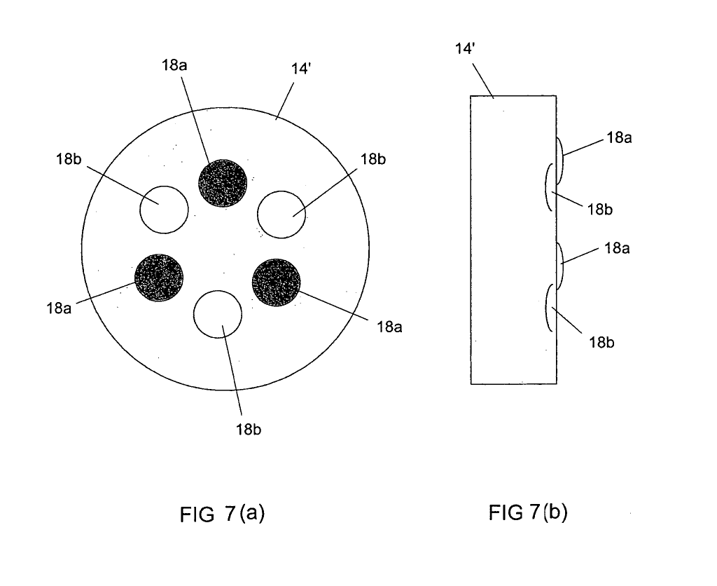

In another embodiment, FIGS. 7(a) and 7(b), the cap 14′ may have an embossed pattern defining a plurality of convex windows 18a alternating with concave windows or depressions 18b. A blanching device incorporating such a cap may measure differences in blood characteristic between adjacent regions of the skin, rather than measuring absolute characteristics for a single region.

The invention is not limited to the embodiments described herein which may be modified or varied without departing from the scope of the invention.

1. A blanching device for use in evaluating skin condition, comprising at least one window carried at the lower end of a support for pressing the window downwardly against the skin to blanch a region of the skin, the window being yieldably connected to the support such that upon reaching a pre-determined blanching pressure the window rapidly retracts upwardly relative to the support to relieve the pressure on the skin and allow blood to re-enter the blanched region.

2. The blanching device of claim 1, wherein the window is provided having a convex shape projecting downwardly from said support.

3. The blanching device of claim 1, wherein the window forms part of a retractable component and the support and the component have interfering formations which resist retraction of the component until the predetermined blanching pressure is reached at which point the resistance is overcome so that the formations snap past each other to allow the component to retract.

4. The blanching device of claim 3, wherein said interfering formations comprise an angular detente provided on the support, and an annular collar provided on the retractable component, the annular collar arranged to abut said angular detente.

5. The blanching device of claim 4, wherein said angular detente is provided on an interior wall of said support, said retractable component arranged to retract into the interior of the support.

6. The blanching device of claim 1, wherein the edges of the window are flexibly connected to the support such that the window retracts by rapidly inverting to a concave shape when the predetermined blanching pressure is reached.

7. The blanching device of claim 1, wherein said support comprises an opaque shroud operable to shield the region of the skin from ambient light.

8. The blanching device of claim 1, wherein a resilient seal portion is provided at the lower end of the support.

9. The blanching device of claim 1, wherein the blanching device comprises a plurality of adjacent windows provided at the lower end of the support for blanching a plurality of adjacent regions of skin.

10. A system for use in evaluating skin condition comprising a blanching device as claimed in claim 1, the system further comprising at least one light emitter to direct light onto the blanched region of skin and at least one light detector to monitor said light after scattering from the blanched region of skin, wherein the light emitter and light detector direct light onto, and receive light from, the blanched region of skin substantially along a common axis which is non-normal to the blanched region of skin so as to substantially eliminate specular reflections from the detected light.

11. The system of claim 10, wherein the system is arranged such that the light is directed onto the blanched region of skin through a window and the common axis is non-normal to the blanched region of skin after refraction by the window, the common axis also being non-normal to the interior surface of the window.

12. The system of claim 10, wherein the light emitter emits light having a wavelength substantially within the range 450 nm-900 nm.

13. The system of claim 1, wherein the system comprises two or more light emitters having different wavelengths of emitted light, said wavelengths substantially within the range 450 nm-900 nm.

14. The system of claim 13, wherein the system comprises first and second light emitters, and wherein the wavelength of the light emitted from the first and second light emitters respectively are substantially within one of the following pairs of ranges: 420 nm-450 nm and 520 nm-580 nm; 600 nm-680 nm and 800 nm-880 nm; or 520 nm-580 nm and βOOnm-680 nm.

15. The system of claim 10, wherein said at least one the light emitter is operated at a switching frequency.

16. The system of claim 15, wherein the switching frequency of the at least one light emitter is substantially within the range 10 Hz-IOO kHz.

17. A system for use in evaluating skin condition, comprising a blanching device as claimed in claim 1, at least one light emitter to direct light onto the blanched region of skin, and at least one light detector to monitor said light after scattering from the blanched region of skin and provide a generally exponential rising signal corresponding to the re-entry of blood into the blanched region, the apparatus further including processing means for analysing the signal into a plurality of components having different time constants, and calculating the proportions of the components in the total signal.

18. The system of claim 17, wherein the processing means is operable to analyse the generally exponential rising signal Z corresponding to the re-entry of blood into the blanched region as the sum of three exponential curves A, B, and C such that:
Z=p.A+q.B+r.C
where p, q and r are coefficients, and p+q+r=1.

19. The system of claim 18, wherein the processing means is operable to select A such that the time constant for A is substantially within the range 0.1-1 second, most preferably about 0.4 seconds.

20. The system of claim 18, wherein the processing means is operable to select B such that the time constant for B is substantially within the range 3-12 seconds, most preferably about 6 seconds.

21. The system of claim 18, wherein the processing means is operable to select C such that the time constant for C is substantially within the range 0.75-2 seconds, most preferably about 1.5 seconds.

22. The system of claim 18, wherein the processing means is operable to calculate a Hyperaemia Indication (HI) value, such that:
(HI)=q(p+q)

23. The system of claim 18, wherein the processing means is operable to calculate an Ischaemic Damage Indication (IDI) value, such that:
(IDI)=p/(p+q)

24. A system for use in evaluating skin condition, comprising means for momentarily pressing on the skin to blanch a region of the skin and then allow blood to re-enter the blanched region, the apparatus further including at least one light emitter to direct light onto the blanched region of skin and at least one light detector to monitor said light after scattering from the blanched region of skin, wherein the light emitter and light detector direct light onto, and receive light from, the blanched region of skin substantially along a common axis which is non-normal to the blanched region of skin so as to substantially eliminate specular reflections from the detected light.

25. A system for use in evaluating skin condition, comprising means for momentarily pressing on the skin to blanch a region of the skin and then allow blood to re-enter the blanched region, at least one light emitter to direct light onto the blanched region of skin, and at least one light detector to monitor said light after scattering from the blanched region of skin and provide a generally exponential rising signal corresponding to the re-entry of blood into the blanched region, the apparatus further including processing means for analysing the signal into a plurality of components having different time constants, and calculating the proportions of the components in the total signal.
